(19) United States
(12) Patent Application Publication
(10) Pub. No.: US 2022/0354562 A1
(43) Pub. Date: Nov. 10, 2022

(54) Cryotherapy Skin Growth Removal Device

(71) Applicants: Amir KATZ, Haifa (IL); Eli BEN SIMCHON, Haifa (IL)

(72) Inventors: Amir KATZ, Haifa (IL); Eli BEN SIMCHON, Haifa (IL)

(21) Appl. No.: 17/780,075

(22) PCT Filed: Nov. 26, 2020
(86) PCT No.: PCT/IB20/61196

§ 371 Date: May 26, 2022

Related U.S. Application Data
(60) Provisional application No. 62/940,741, filed on Nov. 26, 2019.

(51) Int. Cl. A61B 18/02; A61B 18/08

(52) U.S. Cl. CPC A61B 18/02; A61B 18/08; A61B 2018/00452

(57) ABSTRACT

A medical device, including: a cooling system; a heating system; and an applicator including a cold base operationally coupled to the cooling system and a heating element operationally coupled to the heating system, and an applicator head collocated with the cold base and the heating element and adapted for applying a combination of heat and cold to a target area such as target tissue.

### FIELD OF THE INVENTION

The present invention relates to medical devices in general and more specifically to a device for the treatment of skin growths by way of tissue elimination using a combination of cryotherapy and heat therapy.

### BACKGROUND OF THE INVENTION

Conventional devices for tissue elimination using cryotherapy cause necrosis of tissue, both unhealthy tissue as well as surrounding healthy tissue, causing pain and unwanted damage. Another short-coming of conventional devices and methods is that the conventional treatment does not always cause necrosis to the unhealthy tissue.

Often it is necessary to repeat such treatments due to the lack of successful tissue necrosis. Another shortcoming is that the conventional treatment takes a lot of time and is not user friendly and not convenient to use. Yet another shortcoming is that the conventional treatments are expensive, and the equipment is heavy and usually difficult to transport.

### SUMMARY OF THE INVENTION

The present invention is a unique treatment that combines fast gradient of cold and heat transfer. The control of gradients of cooling and heating causes a more effective treatment causing necrosis to the tissue. In addition, the method shortens the time of the treatment. The device is structured and configured so as to control the affect of the treatment on the surrounding area, such that the surrounding tissue will not be adversely affected by the extreme low/high temperatures to which the center of the treatment area is subjected.

Another important feature of the device is the option to combine needles that are inserted into the target/infected tissue causing a more effective necrosis treatment. Moreover, by inserting the needles, the heat transference is much more efficient. Accordingly, the overall treatment is more effective, efficient and takes less time.

According to the present invention there is provided a medical device, including: a cooling system; a heating system; and an applicator including a cold base operationally coupled to the cooling system and a heating element operationally coupled to the heating system, and an applicator head collocated with the cold base and the heating element and adapted for applying a combination of heat and cold to a target area. According to further features in preferred embodiments of the invention described below the target area is a target tissue.

According to still further features in the described preferred embodiments the cooling system is a vapor-compression refrigeration system and possibly a cascade vapor-compression refrigeration system. According to further features the cooling system includes one or more thermoelectric coolers (TEC).

According to further features the cold base and the heating element are in thermal contact with the applicator head, and the cold base is in thermal contact with, and cooled by, tubing adapted to receive cold low-pressure fluid such that when the applicator head comes into contact with the target area, the target area is cryogenically cooled; and wherein the heating element is actuated to provide location specific heat to the target area while the target area is being cooled.

According to further features a disposable component is removably attached to the applicator head, such that the disposable component is adapted to make contact with the target area. According to further features the disposable component is populated with one or more sharp elements, the one or more sharp elements adapted to convey heat and cold into the target area. Exemplarily, the sharp elements are needles.

According to further features alcohol is applied to a face of the disposable component that is adapted to come into contact with the target area. According to further features thermal paste is applied between the applicator head and the disposable component. According to further features the thermal paste is applied between the heating element and the disposable component.

According to further features the heating element is affixed to the applicator head via a slider arrangement, the slider arrangement having a first position wherein the heating element is in contact with the applicator head and a second position wherein the heating element is separated from the applicator head.

According to further features the heating element is disposed in a geometric shape. According to further features the heating element includes more than one heating component. applicator head is an interchangeable applicator head that is removably attached to the applicator.

According to further features the heating system and the cooling system are disposed in a main unit and the applicator is operationally coupled to the main unit via a connector.

According to further features the device further includes a controller configured to provide instructions for controlling a temperature profile of the cooling and heating systems. According to further features the instructions include a number of times for applying the temperature profile.

According to further features the device further includes a communications module adapted to connect to a network to retrieve data regarding methods for implementation of the application of the combination of heat and cold to the target area.

According to further features the device further includes a communications module adapted to connect to a network to update a database regarding instances of application of the combination of heat and cold to the target area.

According to another embodiment there is provided a medical device wherein the heating system, the cooling system and the applicator are disposed in a single unit.

### BRIEF DESCRIPTION OF THE DRAWINGS

Various embodiments are herein described, by way of example only, with reference to the accompanying drawings, wherein:

FIG. 1 is a schematic illustration of a medical device 10 according to the present invention;

FIG. 2 is a diagram of an exemplary cooling system known as a Cascade Mechanical Compression Refrigeration system;

FIG. 3A is an isometric view of applicator 200;

FIG. 3B is a cross-sectional view of applicator 200;

FIG. 3C a cross-sectional view an applicator 200 having a different configuration to the applicator depicted in FIG. 3B;

FIG. 4 is a three-dimensional view of the microchannels 206 of the device;

FIG. 5 is an isometric view of an exemplary disposable sterilized component 214 with needles 215;

FIG. 6A is an exemplary checkered, round contact surface of the heating element 220;

FIG. 6B is an exemplary configuration whereby the heating element 220 is formed in a circular shape around a central area.

### DESCRIPTION OF THE PREFERRED EMBODIMENTS

The principles and operation of a combination cryotherapy device with heating according to the present invention may be better understood with reference to the drawings and the accompanying description.

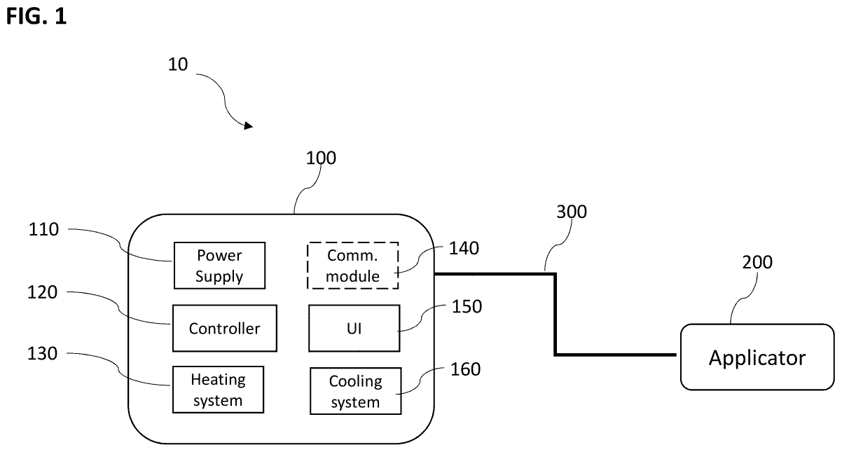

FIG. 1 is a schematic illustration of a medical device 10 according to the present invention. The device 10 consists of a main unit 100 and a handheld applicator 200. In the exemplary embodiment depicted in the Figures, the handheld applicator 200 is coupled to the main unit 100 via a connector 300. Connector 300 includes power and signal cables as well as tubing to carry refrigerant to and from the applicator.

Main unit 100 includes a power supply module 110, a controller 120, a heating system 130, an optional communication module 140, a user interface 150, and a (vapor-compression cascade) cooling system 160.

The power supply module supplies power to the device 110. The power supply module may be connected to power mains. Additionally, or alternatively, the power supply module may include a rechargeable battery. Additionally, the power supply module includes electrical components well known in the art for supplying power to medical devices.

The controller 120 controls all the processes of the device. Controller 120 may be a microcontroller (MCU for microcontroller unit) which is a small computer on a single metal-oxide-semiconductor (MOS) integrated circuit (IC) chip. Exemplarily, the microcontroller may contain one or more CPUs (processor cores) along with memory and programmable input/output peripherals. Program memory in the form of ferroelectric RAM, NOR flash or OTP ROM may also be included on the chip, as well as a small amount of RAM.

In some embodiments, the controller receives and interprets inputs from the input peripherals (e.g. buttons, levers, knobs, dials, a keyboard, mouse, and/or touchscreen console, etc.) of user interface 150 and sends relevant instructions to the applicator. Conversely, signals received from the applicator (e.g. temperature sensor values, etc.) and other components of the main unit are relayed to the controller and relevant information is presented to the user via user interface devices such as a console display, LEDs and the like.

The heating system 130 provides heat to the applicator tip such that the applicator provides both heating and cooling simultaneously. Any type of relevant heating arrangement can be used herein. Accordingly, having a heating system in the main unit may be optional or may merely refer to a heating control system which simply controls a heating element disposed in the handheld applicator. The heating system 140 can control various parameters such as temperature, heating configuration (see below for additional details), power supply, safety parameters etc. Alternatively, the heating system actually generates the heat and transfers the heat to the heating element in the applicator via connector 300.

The communication module 140 facilitates communication between device 10 and external entities. For example, the communication module 140 can facilitate a wired or wireless connection to the Internet. In another example, the communication component can communicate with an external computer (in a wired or wireless manner), e.g. to receive software updates, instructions, protocols and/or to send reports and the like. Any other communication devices can connect to, and interface with, the device 10 via the communications module 140. To this end, the communications module includes components and ports for receiving wired connections to external devices. Additionally, or alternatively, the module includes wireless communication components for connecting and interfacing with external devices in a wireless manner Wired and wireless communication components are well known in the art. The communication module is optional due to the fact that medical device 10 may be a self-contained, stand-alone device that does not communicate with external devices.

User interface 150 includes all the components that the user can use to provide input to the device and to receive output from the device. The user interface can be embodied on a control console in the form of buttons, levers, knobs, dials, a keyboard, mouse/trackball, and/or touchscreen, as is well known in the art. One or more integrated or separate screens can display output information for the user. Other indicators such as blinking and solid lights of various colors can also provide information. Such lights may be embodied in light emitting diodes (LEDs) of different colors.

User interface 150 can also include controls that allow for the activation, deactivation, and modification of various system parameters, such as, for example, gas flow rates, pressures, and temperatures of the refrigerants. The console display can provide the operator the ability to monitor, and in some embodiments adjust, the system to ensure it is performing properly and can provide real-time display as well as recording and historical displays of system parameters.

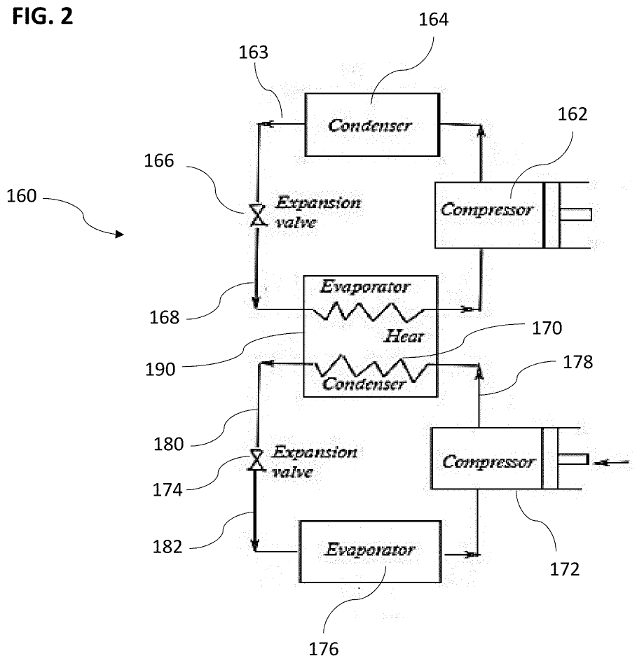

The cooling system 160 is responsible for cooling the tip of the handheld applicator. FIG. 2 illustrates an exemplary cooling system known as a Cascade Mechanical Compression Refrigeration system 160 (also referred to as vapor-compression refrigeration system). The exemplary cascade refrigeration system consists of two compression loops, one for each independent cycle. The exemplary refrigeration system is a freezing system that uses two kinds of refrigerants that have different boiling points. Each coolant runs through an independent freezing cycle, but the cycles intersect at a common heat exchanger.

The first cycle/loop is a High Temperature (HT) cycle and the second cycle is a Low Temperature (LT) cycle. The first cycle has a higher temperature relative to the second cycle. The components of the refrigeration system include: a HT condenser 162, a HT compressor 164, a HT thermal expansion valve 166, and at the low temperature cycle, an LT condenser 170, an LT compressor 172, an LT thermal expansion valve 174, and an LT evaporator 176.

The exemplarily first cycle/loop (shown on top) outputs cold low-pressure liquid/vapor 168 after the cooled high-pressure liquid passes through the expansion valve 166. The cold liquid/vapor cools the hot high-pressure vapor 178 of the second cycle (shown below the first cycle) while passing through heat exchanger 190 in a heat exchange process, effectively cooling the high-pressure vapor 180 of the second cycle to a lower temperature than the parallel cooled (by the condenser 164) high-pressure vapor 163 in the first cycle. As a result, after passing through the expansion valve 174, the cold low-pressure liquid 182 of the second cycle is even colder than the parallel cold low-pressure liquid 168 of the first cycle. Essentially, the cold refrigerant of the first cycle makes the cold refrigerant of the second cycle even colder by making the starting point of the cooled high-pressure liquid temperature lower than it would otherwise be when cooled in ambient temperatures or using a standard fan/condenser such as condenser 164. Evaporator 176 is disposed within the applicator 200, with the refrigerant fluid entering and exiting the applicator from the cooling system via tubing 204 disposed within connector 300.

While the aforementioned system described above uses two different refrigerants with two different boiling points, it is made clear that a similar effect can be achieved using the same refrigerant in both cycles.

Furthermore, while the aforementioned configurations are both effective and relatively cost efficient, other configurations can be employed. For example, a standard single compression cycle refrigeration system can be used instead of the above-described two-cycle cascade system. Alternatively, three or more cycle can be used in a similar cascade configuration to provide even colder temperatures. Whichever configuration is employed, the coldest refrigerant is used to cool the applicator tip for the cryotherapy application.

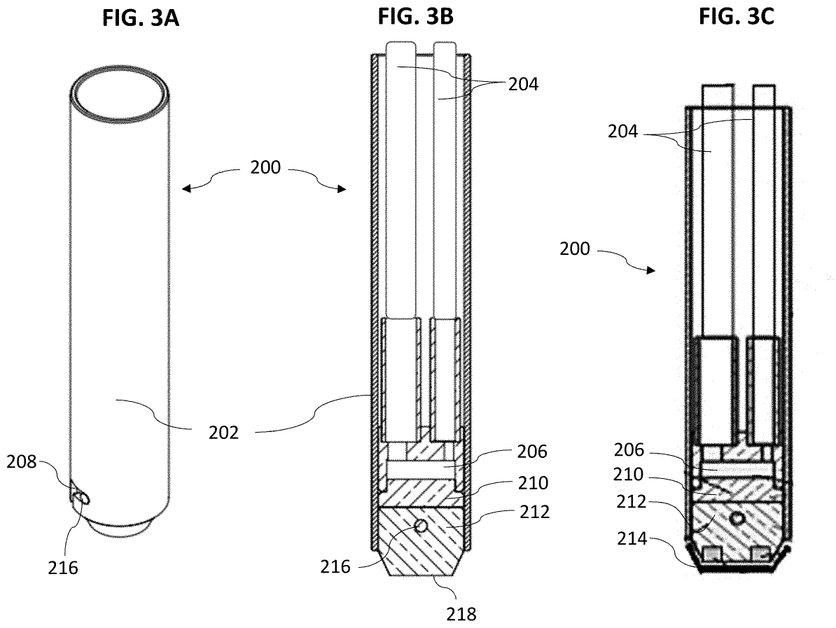

FIGS. 3A and 3B illustrate an exemplary the applicator 200 in greater detail. FIG. 3A depicts an isometric view of applicator 200. FIG. 3B is a cross-sectional view of applicator 200. Applicator 200 is usually a handheld applicator. The applicator consists of a housing 202, applicator tubing 204 and micro channels 206. The liquid flow (e.g. cold, low-pressure refrigerant) entering through the applicator tubing 204, enters the microchannels 206, causing evaporation of the flow and, hence, absorbing heat from the surrounding area. To be clear, the use of microchannels is preferable in order to further increase the cooling potential.

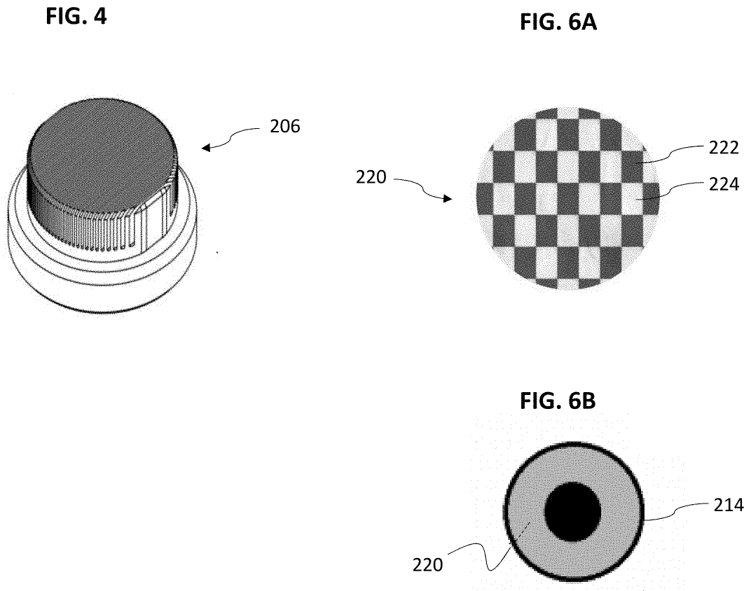

Microchannels are discussed further with reference to FIG. 4. FIG. 4 illustrates a three-dimensional view of the microchannels 206 of the device. Microchannels have large heat transfer surface to volume ratio are cooled with either gas or liquid coolant. By modifying the walls of the microchannel with fins, pins, or grooves, the cooling performance can be improved. A possible fin material used to increase the surface area of a microchannel is carbon nanotubes, which possess excellent thermal and mechanical properties.

Referring back to FIGS. 3A and 3B, the evaporation process occurs through a cold base 210 and an interchangeable applicator head 212. While the exemplary embodiment depicted in the Figures and described here includes an interchangeable applicator head the can be removably placed with a different applicator head, it is made clear that an integrated applicator head that is non-removable is also considered within the scope of the instant innovation.

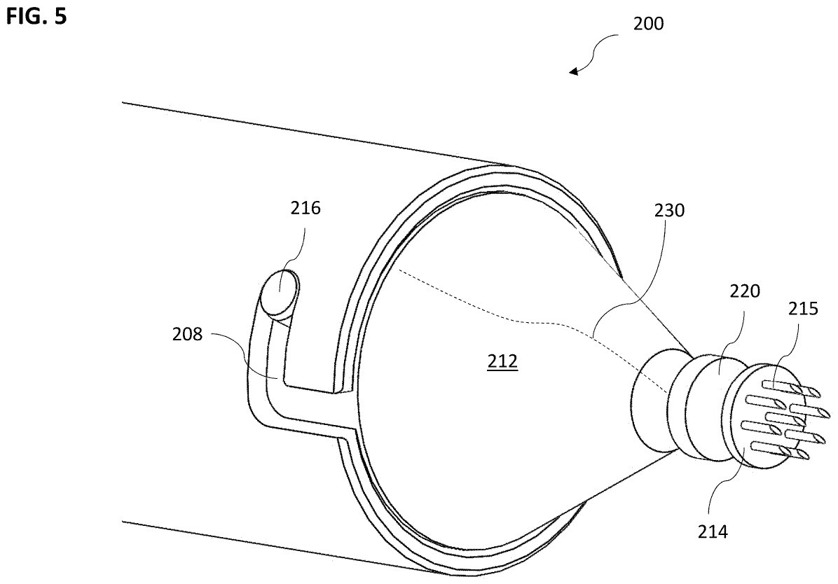

In preferred embodiments, a disposable sterilized component 214 (not shown in FIG. 3B) is detachably attached to the applicator head 212 (see FIG. 3C and FIG. 5). When the disposable component 214 (which is in thermal contact with the applicator head 212, which in turn is in thermal contact with the cold base 210) comes into contact with skin (target tissue), a heat transfer process (evaporation) occurs and the contact causes a temperature of the target tissue to drop down to cryogenic temperatures that are as low as 10° C. and even below −70° C. Heat is transferred to the cold base and conducted to the gas tubing, heating the refrigerant in the tube which cycles back to the cooling system to be re-cooled.

In embodiments without a disposable component, the interchangeable applicator head comes into contact with the target tissue. In such embodiments, a heating element 220 either has a common contact surface with the applicator head or is disposed behind (i.e. internal to the applicator head) the applicator head contact surface 218.

The interchangeable applicator head 212 can come in various sizes and shapes. The applicator head visible best in FIG. 5 is a conical (actually frustoconical). The contact surfaces can be square, rectangular or in fact any shape, as needed. The interchangeable applicator head is attached to the applicator body by sliding a notch 216 into a groove 208 on the applicator housing. The applicator head is preferably made from a material that efficiently and effectively conducts heat.

The applicator 200 further includes a heating element 220. As mentioned, the heating element may be disposed in different locations according to different embodiments of the device. FIG. 3C discloses a cross-sectional view an applicator 200 having a different configuration to the applicator depicted in FIG. 3B. Applicator 200 of FIG. 3C is identical to that of FIG. 3B except for the location of the heating element 220 (behind contact surface 218 and internal to interchangeable applicator head 212).

FIG. 3C further depicts a disposable (i.e. single-use) component 214 which is not shown in FIG. 3B. The disposable sterilized component 214, can be manufactured in different sizes and adjusted according to the desired area and size needed. The disposable component is an interface element between the target tissue and the applicator. Once used on a target tissue (e.g. a wart), the component is preferably discarded for increased sanitation.

Optionally, the disposable sterilized component 214, can be augmented with an alcohol-infused gauze (or other impregnable material) cover which serves as an intermediate fluid layer that improves the heat transfer (from the heating element discussed below). Tangentially, alcohol also acts as a disinfectant to prevent infection of the treated area.

FIG. 5 illustrates an isometric view of an exemplary disposable sterilized component 214 with needles 215. In preferred embodiments component 214 is augmented with one or more sharp elements 215 (e.g. a needle). The sharp element or elements penetrate the treated area for improved heat/cold conduction to the target tissue. Heat and/or cold can be conducted through the sharp element(s) into treated area.

In the instantly depicted embodiment of FIG. 5, the heating element 220 is disposed between the conical applicator head 212 and the disposable component 214. Wires 230 attach the heating element 220 to the heating control system 130 in the main unit 100. Exemplarily, the heating element 220 is affixed to the applicator head via a slider (or similar) arrangement. The slider arrangement (not shown) as a first position whereby the heating element is in contact with the applicator head and a second position wherein the heating element is separated from the applicator head.

Disposable sterilized components 214 can be prepackaged in sealed packaging. In preferred embodiments, the packaging can be foil-lined packages filled with alcohol such that the disposable component is drenched in alcohol for improved thermal-/cryo-conduction. For best results, sharp elements 215 must be located sufficiently close to each other to retain alcohol droplets between the elements. Even though the sharp elements are non-porous, the liquid will not succumb to gravity because of intermolecular forces between the liquid and surrounding solid surfaces (capillarity). The combination of surface tension (which is caused by cohesion within the liquid) and adhesive forces between the liquid and non-porous elements hold the liquid in place.

The heating element 220 may include one or more heating components 222 that can be activated to heat the entire surface or only on parts of the surface. FIGS. 6A and 6B illustrate exemplary embodiments of the heating element 220. Generally, the heating element is disposed between the cold base 210 and the disposable component. In preferred embodiments, thermal paste can be applied between the heating element and the disposable component. Exemplarily, the disposable component may come with pre-applied thermal paste on the device-facing surface of the component. Preferably, the paste is covered over by a non-stick layer that is peeled away prior to connecting the disposable component to the tip of the applicator. Thermal paste is a thermally conductive chemical compound, which is commonly used as an interface between heat sinks and heat sources such as high-power semiconductor devices. The main role of thermal paste is to eliminate air gaps or spaces from the interface area in order to maximize heat transfer and dissipation.

In preferred embodiments, the single-use, disposable component includes legs with protrusions (not shown) for latching the component onto corresponding grooves (not shown) on the applicator head. In some embodiments, the heating element can be mechanically separated from the cold base, e.g. by manipulating a slider arrangement by which the heating element is coupled to the applicator tip/head.

As mentioned, the heating element can be configured to heat the entire surface (e.g. as shown in FIG. 5) or only selected areas. For example, the contact surface can include areas of different shapes for cooling and heating.

Various alternative configurations can be implemented in the heating element. FIG. 6A depicts an exemplary checkered, round contact surface of the heating element 220. The heating element has a checkered configuration where ‘black’ squares are connected to a first heating component/circuit 222 and the ‘white’ squares are connected to a second heating component/circuit 224. The heating system 130 controls the ‘pattern’ of the heating element 220 which is heated. Pattern refers both to the geometrical pattern formed by one or more components 222, 224 as well as the intervals/cycles (if any) at which the geometrical shapes of the heating element 220 are heated. Medical device 10 can cryogenically freeze and thaw the target area multiple times in a short period using the heating element to speed up the thawing process.

There are many ways to implement heating element 220. Exemplary configurations can include one or more heating components 222 adhered to an inner surface 230 of applicator head 212. Heating components 222 can include leads 230 for operable connection to an electrical current source. The heating element may consist of one of more resistors. Alternatively, or additionally, the heating element may be embodied in, or include, radiofrequency (RF) contact. In fact, any heating mechanism, alone or in combination with other heating mechanisms are within the scope of the instant innovation.

In some presently preferred embodiments, the electrical current source can be integral to and located within the heating system 130. Heating components 222 can also connect to a thermal cutoff (not shown), which can also be secured to the inner surface of applicator head 212. If the temperature in the system is increased to an abnormal or unsafe level, the thermal cutoff senses the change and breaks the electrical circuit. A thermocouple (not shown) can also be included to transmit temperature measurements at applicator head 212 through 230 to the main unit 100. The thermocouple can wrap with the heating components 222, 224 or can attach to the inner surface of the applicator head 212 and “float” inside the applicator head 212.

Special Features

The device 10 is specially designed to consistently absorb heat from the treated area while maintaining the cold temperature. The temperature gradient between the cold and hot areas further increases the efficacy of the therapy as the gradient between the heat and cold is very effective in causing necrosis of tissue.

The present invention is described herein as a medical device that treats skin disorders using unique temperature-controlled specifications. However, the instant device can also be used for electrical equipment/plastics/other manufacturing processes and/or for other uses.

According to preferred embodiments the combination heating and cooling is provided simultaneously during application. Alternatively, or additionally, the treatment method employs a process of switching between cooling and heating areas.

According to embodiments, the temperature profile is automatically controlled for both the cooling and heating systems. For example, the device can be set to cool to −70° C. within 3 minutes and rise to 50° C. soon thereafter. Such a process can be rapidly repeated. Cooling and heating cycles such as these can be programmed into the device, e.g. via the user interface or an external device (e.g. running a software application [app] for controlling the device 10). To this end, controller 120 can be configured to provide instructions (a program) for controlling a temperature profile of the cooling and heating systems. The temperature profile is the desired hot and cold parameters, possibly a desired time limit for reaching these temperatures, a desired time during these temperatures are applied to the target area. Furthermore, the instructions include a number of times for applying such a temperature profile.

Another treatment method involves needling during the heating and/or cooling procedure.

Communications module 140 may be adapted to connect to a network (such as the Internet or a private medical network) to retrieve data regarding methods for implementation of the cryotherapy to be used be the instant device for application of the combination of heat and cold to a target area.

Similarly, the communications module 140 may be adapted to connect to a network to update a database regarding instances of application of cryotherapy. The database may be a medical insurance database for purposes of charging an insurer and/or reimbursing a patient.

The instant device 10 is designed to consistently absorb the heat from the treated area while maintaining the cold temperature, unlike other devices where the treated area can influence the device temperature.

The instant device can be employed for hair removal. To this end, the process involves the combination of heating and cooling simultaneously. FIG. 6B depicts an exemplary configuration whereby the heating element 220 is formed in a circular shape around a central area. Accordingly, the center of the disposable component 214 is cooled to an extremely cold temperature (cryo) and the surrounding area is warmed by the heating element 220. The cryo temperature destroys the follicle while the warm areas preserves the skin from being damaged. In preferred implementations, each follicle is identified by image processing methods using a camera or a 3D camera. After identifying the follicle an automatic mechanism moves the applicator head from one follicle to the other. In such an implementation, the applicator is not handheld but rather mounted on a robot arm or other manipulator. The hair-removal method can be used on light hair as well.

In other embodiments the handheld applicator and the main unit may be integrated in a single housing. A single handheld device that includes both the main unit and the applicator may use alternative technologies to cool the applicator tip which take up less space than vapor-compression refrigeration which is described hereafter as part of device 10. One such technology is a Peltier cooler which is a solid-state refrigerator that is small in size and flexible in shape. At present such a cooler suffers drawbacks of high cost for a given cooling capacity (generally less than vapor-compression refrigeration) and poor power efficiency. However, the field of thermoelectric cooling is constantly evolving and improving such that a comparable handheld (i.e. single unit) device is achievable. At present, a single handheld device according to the aforementioned configuration is not as efficient or effective as the above-described vapor-compression based embodiment.

Peltier coolers may also be cascaded into a multi-stage system to achieve lower temperatures. In such an arrangement the hot side of the first peltier cooler is cooled by the cold side of the second peltier cooler, which is larger in size, whose hot side is in turn cooled by the cold side of an even larger peltier cooler, and so on. Efficiency drops very rapidly as more stages are added but for very small heat loads down to near-cryogenic temperatures this can often be an effective solution due to being compact and low cost. All of the aforementioned features, explanations and details apply, mutatis mutandis, to the single-unit embodiment.

It is made clear that any component, mechanism, combination of features, methods and processes disclosed with regards to one embodiment can be equally or similarly applied, mutatis mutandis, to any other embodiment of combination of embodiments.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A medical device, comprising:
a cooling system;
a heating system; and
an applicator including a cold base operationally coupled to said cooling system and a heating element operationally coupled to said heating system, and an applicator head collocated with said cold base and said heating element and adapted for applying a combination of heat and cold to a target area.

2. The device of claim 1, wherein said target area is a target tissue.

3. The device of claim 1, wherein said cooling system is a vapor-compression refrigeration system.

4. The device of claim 1, wherein said cooling system is a cascade vapor-compression refrigeration system.

5. The device of claim 1, wherein said cooling system includes one or more thermoelectric coolers (TEC).

6. The device of claim 3, wherein said cold base and said heating element are in thermal contact with said applicator head, and said cold base is in thermal contact with, and cooled by, tubing adapted to receive cold low-pressure fluid such that when said applicator head comes into contact with said target area, said target area is cryogenically cooled; and
wherein said heating element is actuated to provide location specific heat to said target area while said target area is being cooled.

7. The device of claim 1, wherein a disposable component is removably attached to said applicator head, such that said disposable component is adapted to make contact with said target area.

8. The device of claim 7, wherein alcohol is applied to a face of said disposable component that is adapted to come into contact with said target area.

9. The device of claim 7, wherein said disposable component is populated with one or more sharp elements, said one or more sharp elements adapted to convey heat and cold into said target area.

10. The device of claim 9, wherein said sharp elements are needles.

11. The device of claim 7, wherein thermal paste is applied between said applicator head and said disposable component.

12. The device of claim 11, wherein said thermal paste is applied between said heating element and said disposable component.

13. The device of claim 1, wherein said heating element is affixed to said applicator head via a slider arrangement, said slider arrangement having a first position wherein said heating element is in contact with said applicator head and a second position wherein said heating element is separated from said applicator head.

14. The device of claim 1, wherein said heating element is disposed in a geometric shape.

15. The device of claim 1, wherein said heating element includes more than one heating component.

16. The device of claim 1, wherein said applicator head is an interchangeable applicator head that is removably attached to said applicator.

17. The device of claim 1, wherein said heating system and said cooling system are disposed in a main unit and said applicator is operationally coupled to said main unit via a connector.

18. The device of claim 1, wherein said heating system, said cooling system and said applicator are disposed in a single unit.

19. The device of claim 9, wherein a plurality of said sharp elements are disposed in sufficient proximity to each other to support capillarity there-between.

20. The device of claim 1, further comprising a controller configured to provide instructions for controlling a temperature profile of said cooling and heating systems.

21. The device of claim 20, wherein said instructions include a number of times for applying said temperature profile.

22. The device of claim 1, further comprising a communications module adapted to connect to a network to retrieve data regarding methods for implementation of said application of said combination of heat and cold to said target area.

23. The device of claim 1, further comprising a communications module adapted to connect to a network to update a database regarding instances of application of said combination of heat and cold to said target area.
